(19) United States
(12) Patent Application Publication
(10) Pub. No.: US 2008/0306313 A1
(43) Pub. Date: Dec. 11, 2008

(54) Non Thermal Plasma Method to Prepare [11C] Methyl Iodide From [11C] Methane and Iodine Methane and Iodine

(76) Inventors: Jonas Eriksson, Amsterdam (NL); Tor Kihlberg, Uppsala (SE); Bengt Langstrom, Uppsala (SE); Johan Ulin, Uppsala (SE)

Correspondence Address: GE HEALTHCARE, INC., IP DEPARTMENT, 101 CARNEGIE CENTER, PRINCETON, NJ, 08540-6231, US

(21) Appl. No.: 12/097,563

(22) PCT Filed: Dec. 12, 2006
(86) PCT No.: PCT/IB06/03588

§ 371 Date: Jun. 16, 2008

Related U.S. Application Data
(60) Provisional application No. 60/750,132, filed on Dec. 14, 2005.

(51) Int. Cl. C07C 17/35

(52) U.S. Cl. 570/255

(57) ABSTRACT

A method and an apparatus for preparing [¹¹C]methyl iodide from [¹¹C]methane and iodine in a single pass through a non-thermal plasma reactor has been developed. The plasma was created by applying high voltage (400 V/31 kHz) to electrodes in a stream of helium gas at reduced pressure. The [¹¹C]methane used in the experiments was produced from [¹¹C]carbon dioxide via reduction with hydrogen over nickel. [¹¹C]Methyl iodide was obtained with a specific radioactivity of 412±32 GBq/μmol within 6 min from approximately 24 GBq of [¹¹C]carbon dioxide. The decay corrected radiochemical yield was 13±3% based on [¹¹C]carbon dioxide at start of synthesis. [¹¹C]Flumazenil was synthesized via a N-alkylation with the prepared [¹¹C]methyl iodide. Kit and apparatus for preparing [¹¹C]methyl iodide by a non thermal plasma method are also provided.

### FIELD OF THE INVENTION

The present invention relates to a method and apparatus of preparing [¹¹C]methyl iodide from [¹¹C]methane and iodine in a single pass through a non-thermal plasma reactor system. The present invention further relates to kits for producing a method and apparatus of [¹¹C]methyl iodide in a non thermal plasma reactor system. [¹¹C]methyl iodide is a precursor in the synthesis of ¹¹C-labeled Positron Emission Tomography (PET) tracers.

### BACKGROUND OF THE INVENTION

The advances in non-thermal plasma chemistry may open for new methods to convert cyclotron produced ¹¹C-labelled compounds such as [¹¹C]carbon dioxide or [¹¹C]methane to other useful precursors. Some applications of non-thermal plasma are surface treatment, fluorescent lightning and flat screens. Wagner H E, Brandenburg R, Kozlov K V, Sonnenfeld A, Michel P, Behnke J F. Vacuum 2002; 71: 417-436; Kogelschatz U. Plasma Chem Plasma Process 2003; 23: 1-46. Ozone is produced with non-thermal plasma at industrial scale and complex organic compounds have been synthesised in good yield. Suhr H. Plasma Chem Plasma Process 1983; 3: 1-61. Plasma techniques have also been applied in the synthesis of ¹¹C-labelled compounds. Crouzel C, Sejourne C, Comar D. Int J Appl Radiat Isot 1979; 30: 566-568; Niisawa K, Ogawa K, Saito T, Taki K, Karasawa T, Nozaki T. Int J Appl Radiat Isot 1984; 35: 29-33. Recently, progress has been made in developing methods for the conversion of methane to low-weight hydrocarbons. Eliasson B, Liu C, Kogelschtz U. Ind Eng Chem Res 2000; 39: 1221-1227; Liu C J, Xu G H, Wang T. Fuel Process Technol 1999; 58: 119-134. Okumoto et al showed that methane in the presence of oxygen was converted to a variety of products via a single pass through a dielectric barrier discharge plasma reactor. Okumoto M, Kim H H, Takashima K, Katsura S, Mizuno A. IEEE T Ind Appl 2001; 37: 1618-1624. The selectivity of the reaction was approximately 30% with respect to methanol. Methyl iodide was obtained with a selectivity of 95% when oxygen was replaced with iodine. Okumoto M, Mizuno A. Catal Today 2001; 71: 211-217. No methylene iodide or iodoform was detected. These findings inspired Applicants to explore the possibility to convert [¹¹C]methane to other useful precursors by use of non-thermal plasma.

A group of diagnostic Positron Emission Tomography (PET) procedures utilize radioactive labeled compounds, wherein the radioactive atoms are positron emitters. Some examples of positron emitting elements include nuclides of carbon, nitrogen, or fluorine. These elements are the backbone of almost all biological active compounds. In order to be able to use these elements, stable isotopes are replaced with a radioactive isotope. The radioactive labeled compounds, called tracers, are transported, accumulated and converted exactly the same way as for non-radioactive compounds. The PET method has possibilities to detect malfunction on a cellular level in the investigated tissues or organs. The method is very sensitive and requires only nanomole quantities of produced radioactive tracers. These radioactive tracers have a half-life in the range from 2 to 110 minutes, (e.g. ¹¹C, t_{1/2}=20.3 min). Because of the radioactivity, the short half-lives and the submicromolar amounts of the labeled substances, extraordinary synthetic procedures are required for the production of these tracers. An important part of the elaboration of these procedures is the development and handling of new ¹¹C-labeled precursors. This is important not only for labeling new types of compounds, but also for increasing the possibility of labeling a given compound in different positions.

One important and very useful starting compound is carbon-11 labeled methyl iodide. At present [¹¹C]methyl iodide is the most commonly used precursor in synthesis of ¹¹C-labeled PET-tracers. The main reason is that hetero atom (e.g. N, O, S) bound methyl groups, that are easy to label with [¹¹C]methyl iodide, are common among biologically active compounds such as endogenous and pharmaceutical substances. With ¹¹C-labeled methyl iodide it is possible to make a large variety of ¹¹C-labeled compounds. These are of interest for diagnosis and follow up of a treatment of, for example, cancer, epilepsy, or dementia.

Such a compound is most often formed from ¹¹C-labeled carbon dioxide through reduction with lithium aluminum hydride (LAH) to ¹¹C-labeled methanol and a reaction of this compound with hydrogen iodide to produce ¹¹C-labeled methyl iodide. The reaction takes place in an organic solvent. This method has several disadvantages; the chemicals are cumbersome to use which makes the process unreliable and the LAH contains a variable amount of cold carbon dioxide lowering the relation between produced radioactive and non-radioactive ¹¹C-labeled methyl iodide. In many cases it is desirable to have a high ratio.

Another way to produce ¹¹C-labeled methyl iodide is the halogenation of ¹¹C-labeled methane with iodine. The ¹¹C-labeled methane is formed from the catalytic reduction of ¹¹C-labeled carbon dioxide. The halogenation reaction of the ¹¹C-labeled methane is a non-selective radical reaction taking place under elevated temperatures. As iodine always will be present in large excess it is difficult to prevent polyhalogenation, leading to low radiochemical purity. A wet phase method was used to obtain ¹¹C-labeled methyl iodide in Langstrom et al. “The Preparation of ¹¹C-Methyl Iodide and its Use in the Synthesis of ¹¹C-Methyl-_{L}-Methionine”, Langstrom et al., International Journal of Applied Radiation and Isotopes, 1976, vol. 27, pp. 357-363. However, the problem of obtaining a low radio chemical purity of the radical ionization reaction has recently been solved by Larsen et al. by using a gas phase method to obtain ¹¹C-labeled methyl iodide. U.S. Pat. No. 6,008,421.

Additionally, recent developments in the direct conversion of natural gas and its principal component methane to a liquid product such as methanol and formaldehyde in homogeneous gas-phase reactions, and over heterogeneous catalysts have led to developments in investigating the methanol synthesis from methane and O₂ using a pulsed discharge plasma process. “Conversion of methane for higher hydrocarbon fuel synthesis using pulsed discharge plasma method”, Okumoto et al., Catalyst Today, 2001, vol. 71, pp. 211-217). The pulse plasma discharge method was used to control the methane oxidation and to produce methanol and formaldehyde as intermediate chemicals in the oxidation of methane. Id. Furthermore, to prevent excessive oxidations such as production of methyl components and to improve the production selectivity, the addition of halogen materials have been investigated. Id.

In a secondary consideration, Okumoto et al. investigated the halogenation of methane in order to protect the products chemically, to avoid a further reaction of intermediate products in the pulse plasma discharge region. Moreover, the halogenation of methane has been investigated for selective partial oxidation of methane since the 1960s. Some cases showed good performance to achieve partial oxidation of methane with catalysis. Asahi-Kasei Co., Japanese Patent No. 478,295 (1966). However, the systems used prior to the introduction of a pulse discharge method were hindered by the fact that halogenide works as a catalytic poison. On the contrary, in the case of a pulsed discharge method for halogenation of methane, the achievement of partial oxidation of methane was hardly affected by the presence of halogenide. Moreover, the pulsed discharge plasma method was carried out with an experiment using the halogenation of methane to methyl halide. Okumoto et al. As a result of this experiment, it was shown that chemical protection by halogenide enhanced the reaction selectivity of methane to methanol is possible using the halogenation of methane. Id. Furthermore, other researchers showed that hydrolysis of methyl iodine occurred when alkyl solvent and methanol was produced. “Novel, High-yield System for the Oxidation of methane to Methanol, Electron Transfer Reactions: Inorganic, Organometallic, Biological Applications”, R. A. Periana, American Chemical Society, Washington, D.C., 1997, pp. 61-78.

Carbonylation reactions using [¹¹C]methane have a primary value for PET-tracer synthesis since biologically active substances often contain carbonyl groups or functionalities that can be derived from a carbonyl group. The syntheses are tolerant to most functional groups, which means that complex building blocks can be assembled in the carbonylation step to yield the target compound. This is particularly valuable in PET-tracer synthesis where the unlabeled substrates should be combined with the labeled precursor as late as possible in the reaction sequence, in order to decrease synthesis-time and thus optimize the uncorrected radiochemical yield.

Accordingly, there is a need for creating a rapid synthetic route that could be used for labeling several compounds of biological interest using ¹¹C-labeled methyl iodide. This rapid synthetic route can be achieved by the iodination of [¹¹C]methane in a single pass through a non-thermal plasma reactor containing iodine vapour to produce [¹¹C]methyl iodide is described.

Discussion or citation of a reference herein shall not be construed as an admission that such reference is prior art to the present invention.

### SUMMARY OF THE INVENTION

In view of the needs of the prior art, the present invention provides a method of obtaining [¹¹C]methyl iodide from [¹¹C]methane and iodine in a plasma reactor system. The plasma reactor is turned on and off in a similar fashion as a fluorescent lamp and generates no heat.

Unlike previous methods, [¹¹C]methane is obtained from the reduction of [¹¹C]carbon dioxide with nickel and hydrogen gas or directly from a cyclotron methane target, wherein the [¹¹C]methane is carried in a stream of noble gas. After passing a carbon dioxide trap (ascarite) and a drying tower (phosphorous pentoxide), [¹¹C]methane is concentrated on porapac at −196 C. The carbon dioxide trap is flushed with helium to remove hydrogen. A hydrogen trap (Pd/Al₂O₃) may be used to remove any residual hydrogen. A mass flow regulator sets the mass flow and marks the start of the low pressure area of the system. The low pressure area contains an iodine source, a plasma reactor and an iodine trap (wet ascarite). An online pump regulates the pressure. [¹¹C]methane is transferred via the iodine feed to the plasma reactor. Iodine vapors evolve due to the reduced pressure and passes through the plasma reactor simultaneously with the [¹¹C]methane. A voltage (400 V, 31 kHz) is applied to the electrodes positioned in the gas stream inside the plasma reactor. Electrons are stripped from the carrier gas, the conductivity is increased and plasma is created. Thereafter, [¹¹C]methyl iodide is formed in chemical reactions initiated by the ionization of methane, and is transferred via an iodine trap and the online pump, to a reaction vial containing a suitable solvent or a loop reaction system. The plasma herein is attained by applying a high voltage, about 400 Volts, to the electrodes positioned inside the plasma reactor wherein the plasma is carried in a stream of carrier gas at regulated pressure and mass flow into the plasma reactor.

The present invention depicts a method of preparing [¹¹C]methyl iodide in a plasma reactor system, comprising the steps of:
obtaining methane (A1) from the reduction of carbon dioxide with nickel and then adding a cyclotron to produce [¹¹C] onto the methane target whereby the [¹¹C]methane is carried into said system in a stream of a carrier gas whereby a carbon dioxide trap (A2), a drying tower (A2 and A4), and a hydrogen trap (A3) are used to remove hydrogen;
introducing a mass flow regulator (A5) to mark the start of the low pressure area of said system whereby the low pressure area contains an iodine source (A6), a plasma reactor (A7), and a trapping chamber (A8) and wherein the mass flow regulator is regulated by a pump (A10);
transferring [¹¹C]methane via said iodine source into said plasma reactor, whereby iodine vapors form and pass through said system simulataneously with [¹¹C]methane to further form a gas stream;
applying a voltage to electrodes positioned in said gas stream inside said system, whereby electrons are stripped from the carrier gas and plasma is created thus forming a [¹¹C]methyl iodide plasma, and then
transferring said [¹¹C]methyl iodide plasma via said trapping chamber and said pump to a reaction vial (A11) containing a suitable solvent or a loop reaction system.

In a further embodiment, the present invention shows an apparatus for preparing [¹¹C]methyl iodide in a plasma reactor system, comprising [¹¹C]methane produced from the reduction of carbon dioxide with nickel and a cyclotron wherein said system further comprises a stream of a [¹¹C]methane carrier gas whereby a carbon dioxide trap (A2), a drying tower (A2 and A4), and a hydrogen trap (A3) are used to remove hydrogen wherein said system further comprises a mass flow regulator (A5) where within contains an iodine source (A6), a plasma reactor (A7), and a trapping chamber (A8) and wherein the mass flow regulator is regulated by a pump (A10) the system further comprises a voltage that is applied to electrodes positioned in said gas stream, and the system further comprises a [¹¹C]methyl iodide plasma and a reaction vial (A11).

Yet another embodiment comprises a kit for preparing a method for producing [¹¹C]methyl iodide, wherein the kit comprises the steps of:
obtaining methane (A1) from the reduction of carbon dioxide with nickel and then adding a cyclotron to produce [¹¹C] onto the methane target whereby the [¹¹C]methane is carried into said system in a stream of a carrier gas whereby a carbon dioxide trap (A2), a drying tower (A2 and A4), and a hydrogen trap (A3) are used to remove hydrogen;
introducing a mass flow regulator (A5) to mark the start of the low pressure area of said system whereby the low pressure area contains an iodine source (A6), a plasma reactor (A7), and a trapping chamber (A8) and wherein the mass flow regulator is regulated by a pump (A10);
transferring [¹¹C]methane via said iodine source into said plasma reactor, whereby iodine vapors form and pass through said system simulataneously with [¹¹C]methane to further form a gas stream;
applying a voltage to electrodes positioned in said gas stream inside said system, whereby electrons are stripped from the carrier gas and plasma is created thus forming a [¹¹C]methyl iodide plasma, and then
transferring said [¹¹C]methyl iodide plasma via said trapping chamber and said pump to a reaction vial (A11) containing a suitable solvent or a loop reaction system.

The present invention also provides a kit for preparing an apparatus for producing [¹¹C]methyl iodide, wherein the kit comprises an effective amount of [¹¹C]methyl iodide plasma by reducing methane from carbon dioxide, nickel and a cyclotron wherein said system further comprises a stream of a [¹¹C]methane carrier gas whereby a carbon dioxide trap (A2), a drying tower (A2 and A4), and a hydrogen trap (A3) are used to remove hydrogen wherein said system further comprises a mass flow regulator (A5) where within contains an iodine source (A6), a plasma reactor (A7), and a trapping chamber (A8) and wherein the mass flow regulator is regulated by a pump (A10) the system further comprises a voltage that is applied to electrodes positioned in said gas stream, and the system further comprises a [¹¹C]methyl iodide plasma and a reaction vial (A11).

### BRIEF DESCRIPTION OF FIGURES

The invention will be described by preferred embodiments to be contemplated with reference to the accompanying drawings wherein like reference numerals are used throughout to designate like parts. In the drawings:

FIG. 1 shows an embodiment of a plasma methyl iodide system including an on-line pump that controls the mass regulator of [11C]methane that is carried into the plasma reactor system in a stream of a carrier gas such as a noble gas.

FIG. 2 depicts another embodiment of a plasma methyl iodide system including an off-line pump that controls the mass regulator of [11C]methane that is carried into the plasma reactor system in a stream of a carrier gas such as a noble gas.

FIG. 3 presents a plasma reactor used in the plasma methyl iodide system. The plasma reactor is encased in quartz glass and Wolfram electrodes are used in side the plasma reactor to aid in creating plasma.

### DETAILED DESCRIPTION OF THE INVENTION

[¹¹C]methyl iodide is a precursor in the synthesis of several Positron Emission Tomography (PET)-tracers. Two production methods that are available today are the so called “wet method” and the “gas phase method”. The most similar of the two methods in comparison to the current invention is the gas phase method. The gas phase method is a method that utilizes a recirculating system where [¹¹C]methane and iodine vapours are reacted at high temperatures such as 500 degrees Celcius.

The current invention sets forth several advantages over the gas phase method and the wet phase method. The current non thermal plasma method presents an ease of use over the other two methods, a quicker time for the production of [¹¹C]methyl iodide, a non thermal plasma reactor, and a shorter total synthesis and cycle time. These parameters determine the maximum number of productions that can be made over a given time period. This is important for efficiently supplying a PET center with radioactive precursor batches needed for synthesis of tracers for PET-scans. Short synthesis times will also yield compounds with higher radiochemical yield and specific radioactivity (Becquerel/mole) due to less decay. Radiochemical purity (RCP) is defined as the amount of radioactivity originating from a specific substance in relation to the total amount of radioactivity in a sample, expressed in %. Additionally, specific radioactivity (Becquerel/mole) is the ratio between the amount of radioactivity originating from a specific substance labeled with a radionuclide and the total amount of that specific substance.

Additional characteristics that are important factors favoring the non thermal plasma method over the gas phase method include: miniaturization of the apparatus; selection of a carrier gas such as helium, neon, argon, or the like; quicker mass flow rates through the plasma reactor in about 1 ml/min to about 50 ml/min; very low pressure in the plasma reactor of about 5 mbar to about 250 mbar; a quick gas residence time in reactor of about 0.6 to about 1.0 seconds; amount iodine released to the reactor from about 50 mg to about 250 mg; type of methane trap; removal of hydrogen gas and other unwanted contaminants; concentration of the radioactive gas mixture fed to the reactor; power supply voltage and frequency. Different plasma reactors could also improve the gas phase method as well as the general design of the system.

Furthermore, an iodide trap or the trapping chamber in said invention is a column containing a material that efficiently absorbs iodide but not methyl iodide. In the described set up Ascarite® (i.e. sodium hydroxide on silica) was used. A hydrogen trap is a column containing a material that selectively absorbs hydrogen. The material could be a finely dispersed transition metal on a high surface matrix such as silica. The material used as hydrogen trap in the working example is palladium, 5% on 3 mm alumina pellets. Additionally, a methane trap is a column containing a material that selectively absorbs methane at a certain temperature (e.g. −196° C.) and releases the trapped methane at a higher temperature with high recovery in both steps. In the described set up the GC-material Porapac Q® was used. Due to the difference in boiling point between hydrogen and methane and the difference in van der Waal interaction between hydrogen and methane versus the Porapac Q® material a selective removal of hydrogen can be obtained during the trapping.

Below a detailed description is given of a method for producing [¹¹C]methyl iodide from [¹¹C]methane through a non thermal plasma reactor system. The present invention also relates to an apparatus of preparing a non thermal plasma reactor system. The present invention further relates to kits for producing a method and apparatus of [¹¹C]methyl iodide in a non thermal plasma reactor system.

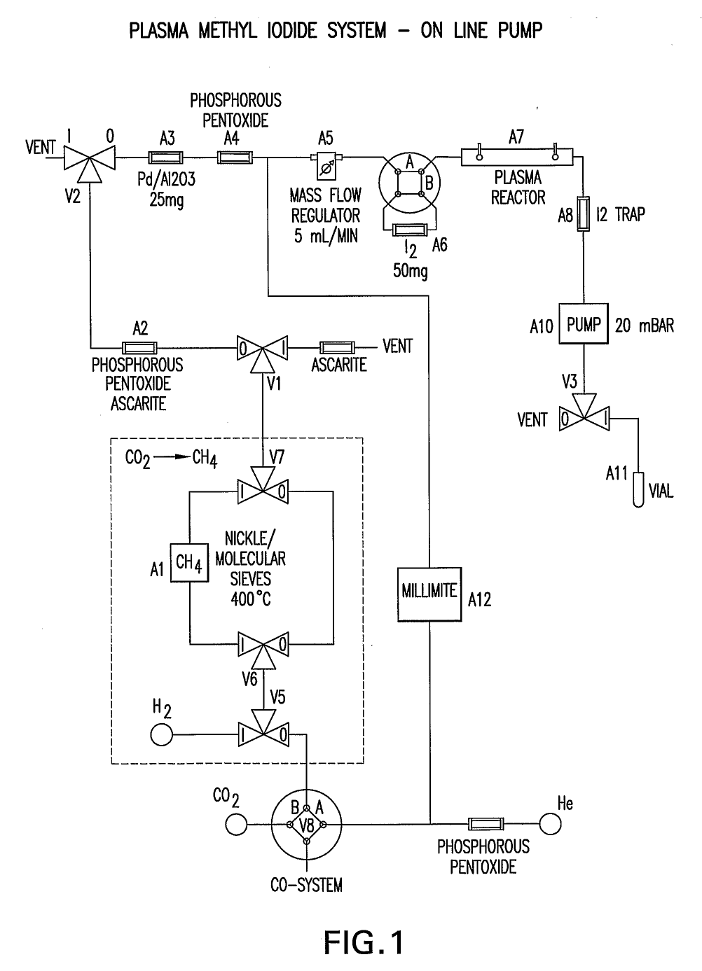

In one embodiment of the present invention a method for preparing [¹¹C]methyl iodide in a plasma reactor system is introduced. Preparing [¹¹C]methyl iodide comprising the steps of:
obtaining methane (A1), from the reduction of carbon dioxide with nickel and then adding a cyclotron to produce [¹¹C] onto the methane target, wherein the concentration range of [¹¹C]methane is less than 1⁻⁹ mole, whereby [¹¹C]methane is carried into said system in a stream of a carrier gas whereby a carbon dioxide trap (A2), a drying tower (A2 and A4), and a hydrogen trap (A3) are used to remove hydrogen; further wherein a cyclotron is used for the production of ¹¹C and a gas target is filled with nitrogen gas and hydrogen gas (typically 0.1-5%). The gas is irradiated with protons (commonly with the energy of approximately 12-40 MeV) through a thin metal foil window in the gas target. The nuclear reaction ¹⁴N (P,α) ¹¹C occurs. The ¹¹C-species produced reacts with the hydrogen present in the target and [¹¹C]methane is formed.
introducing a mass flow regulator (A5), wherein the mass flow is the mass of gas per unit of time flowing in a closed fluid channel, to mark the start of the low pressure area of said system whereby the low pressure area contains an iodine source (A6), a plasma reactor (A7), and a trapping chamber (A8) and wherein the mass flow regulator is regulated by a pump (A10); furthermore, if the pump capacity of the vacuum pump (A10) is higher than the set value of the mass flow regulator, there will be a “low pressure” area between the flow regulator and the pump. Independently of how low the pressure gets in this area the mass flow regulator will keep the mass per unit of gas flowing through the regulator constant according to the set value. This feature is used to control the gas flow and the pressure in the plasma reactor. Furthermore, the pump (A10) should have sufficient pump capacity to lower the pressure in the plasma reactor to 50-250 mbar. If the pump is used “on line” as seen in FIG. 1, it should have a gas tight construction which does not let air or other contaminants, it should not contaminate the gas for example with oil residues and it should have good chemical resistance (iodine vapors mainly).
transferring [¹¹C]methane via said iodine source into said plasma reactor, whereby iodine vapors form and pass through said system simulataneously with [¹¹C]methane to further form a gas stream;
applying a voltage to electrodes positioned in said gas stream inside said system, whereby electrons are stripped from the carrier gas and plasma is created thus forming a [¹¹C]methyl iodide plasma, wherein less voltage is needed if either the electrodes are closer to each other or the pressure in the plasma reactor is lowered, and then
transferring said [¹¹C]methyl iodide plasma via said trapping chamber and said pump to a reaction vial (A11) containing a suitable solvent or a loop reaction system.

In a further embodiment of the present invention, the pump (A10) pressure is 5-25 bar. The plasma reactor pressure is dependent on pump pressure, setting of the mass flow regulator and flow restrictions between the reactor and pump. The optimal pressure in the reactor is estimated to be around 100 mbar.

Yet in another embodiment of the invention, the drying tower is phosphorous pentoxide and the hydrogen trap is Pd/Al₂O₃.

In another embodiment, the carrier gas is a noble gas such as helium, argon, and neon. The carrier gas residence time in the plasma reactor system is in the range from about 0.5-1.0 second.

In a further embodiment, the plasma reactor generates no heat.

Yet in another embodiment, the iodine source comprises of about 50 mg of I₂ and the trapping chamber is a column containing sodium hydroxide on silica that absorbs iodide but not methyl iodide. The trapping chamber further comprises an optional CH₃I-trap.

In another embodiment, the pump is positioned on-line or off-line.

In a further embodiment, the voltage applied to the electrodes positioned in said gas stream is about 400 Volts with a frequency of about 31 kHz.

In another embodiment, the mass flow regulator has a mass flow of about 1 ml/min to about 50 ml.min.

In a further embodiment, the suitable solvents comprises compounds that have high boiling points in the range of 270 Kelvin to 380 Kelvin. Examples of suitable solvents are dimethyl sulfoxide, N,N-dimethyl formamide, N-methyl pyrollidone, or similar compounds.

Another embodiment of the present invention is wherein the loop reaction system is a method for using small amounts of reaction media to trap the [¹¹C]methyl iodide, whereby the reaction media is coated on the internal surface of a piece of tubing and the [¹¹C]methyl iodide is then directed through the tubing to get trapped in the reaction media. The reaction media is [¹¹C]methane.

In another embodiment, any unreacted [¹¹C]methane is recirculated back into the plasma reactor and the [¹¹C]methyl iodide would be taken out from the recirculation by the use of a methyl iodide trap.

In another embodiment of the present invention, an apparatus for preparing [¹¹C]methyl iodide in a plasma reactor system is claimed. The apparatus comprises [¹¹C]methane produced from the reduction of carbon dioxide with nickel and a cyclotron wherein said system further comprises a stream of a [¹¹C]methane carrier gas whereby a carbon dioxide trap (A2), a drying tower (A2 and A4), and a hydrogen trap (A3) are used to remove hydrogen wherein said system further comprises a mass flow regulator (A5) where within contains an iodine source (A6), a plasma reactor (A7), and a trapping chamber (A8) and wherein the mass flow regulator is regulated by a pump (A10) the system further comprises a voltage that is applied to electrodes positioned in said gas stream, and the system further comprises a [¹¹C]methyl iodide plasma and a reaction vial (A11).

Another embodiment presents a kit for preparing a method for producing [¹¹C]methyl iodide, wherein the kit comprises the steps of:
obtaining methane (A1) from the reduction of carbon dioxide with nickel and then adding a cyclotron to produce [¹¹C] onto the methane target whereby the [¹¹C]methane is carried into said system in a stream of a carrier gas whereby a carbon dioxide trap (A2), a drying tower (A2 and A4), and a hydrogen trap (A3) are used to remove hydrogen;
introducing a mass flow regulator (A5) to mark the start of the low pressure area of said system whereby the low pressure area contains an iodine source (A6), a plasma reactor (A7), and a trapping chamber (A8) and wherein the mass flow regulator is regulated by a pump (A10);
transferring [¹¹C]methane via said iodine source into said plasma reactor, whereby iodine vapors form and pass through said system simulataneously with [¹¹C]methane to further form a gas stream;
applying a voltage to electrodes positioned in said gas stream inside said system, whereby electrons are stripped from the carrier gas and plasma is created thus forming a [¹¹C]methyl iodide plasma, and then
transferring said [¹¹C]methyl iodide plasma via said trapping chamber and said pump to a reaction vial (A11) containing a suitable solvent or a loop reaction system.

The present invention also provides for a kit for preparing an apparatus for producing [¹¹C]methyl iodide, wherein the kit comprises an effective amount of [¹¹C]methyl iodide plasma by reducing methane from carbon dioxide, nickel and a cyclotron wherein said system further comprises a stream of a [¹¹C]methane carrier gas whereby a carbon dioxide trap (A2), a drying tower (A2 and A4), and a hydrogen trap (A3) are used to remove hydrogen wherein said system further comprises a mass flow regulator (A5) where within contains an iodine source (A6), a plasma reactor (A7), and a trapping chamber (A8) and wherein the mass flow regulator is regulated by a pump (A10) the system further comprises a voltage that is applied to electrodes positioned in said gas stream, and the system further comprises a [¹¹C]methyl iodide plasma and a reaction vial (A11).

The present invention further provides a method of use for preparing [¹¹C]methyl iodide in a plasma reactor system, comprising the steps of:
obtaining methane (A1) from the reduction of carbon dioxide with nickel and then adding a cyclotron to produce [¹¹C] onto the methane target whereby [¹¹C]methane is carried into said system in a stream of a carrier gas whereby a carbon dioxide trap (A2), a drying tower (A2 and A4), and a hydrogen trap (A3) are used to remove hydrogen;
introducing a mass flow regulator (A5) to mark the start of the low pressure area of said system whereby the low pressure area contains an iodine source (A6), a plasma reactor (A7), and a trapping chamber (A8) and wherein the mass flow regulator is regulated by a pump (A10);
transferring [¹¹C]methane via said iodine source into said plasma reactor, whereby iodine vapors form and pass through said system simulataneously with [¹¹C]methane to further form a gas stream;
applying a voltage to electrodes positioned in said gas stream inside said system, whereby electrons are stripped from the carrier gas and plasma is created thus forming a [¹¹C]methyl iodide plasma, and then
transferring said [¹¹C]methyl iodide plasma via said trapping chamber and said pump to a reaction vial (A11) containing a suitable solvent or a loop reaction system.

The present invention also provides for the use of an apparatus for preparing [¹¹C]methyl iodide in a plasma reactor system, comprising [¹¹C]methane produced from the reduction of carbon dioxide with nickel and a cyclotron wherein said system further comprises a stream of a [¹¹C]methane carrier gas whereby a carbon dioxide trap (A2), a drying tower (A2 and A4), and a hydrogen trap (A3) are used to remove hydrogen wherein said system further comprises a mass flow regulator (A5) where within contains an iodine source (A6), a plasma reactor (A7), and a trapping chamber (A8) and wherein the mass flow regulator is regulated by a pump (A10) the system further comprises a voltage that is applied to electrodes positioned in said gas stream, and the system further comprises a [¹¹C]methyl iodide plasma and a reaction vial (A11).

### EXAMPLES

The invention is further described in the following examples which are in no way intended to limit the scope of the invention.

Experimental

General

¹¹C was prepared by the ¹⁴N(p,α)¹¹C nuclear reaction using 17 MeV protons produced by a Scanditronix MC-17 Cyclotron at Uppsala Imanet AB and obtained as [¹¹C]carbon dioxide. The target gas used was nitrogen (AGA Nitrogen 6.0) containing 0.05% oxygen (AGA Oxygen 4.8). [¹¹C]Carbon dioxide was transferred in a stream of nitrogen gas from the cyclotron to the reduction reactor containing a mix of molecular sieves 4 Å60/80 and nickel powder 50:50 w %.

Procedure for Converting [¹¹C]Carbon dioxide to [¹¹C]Methyl Iodide Using the Single Pass Non-Thermal Plasma System, FIG. 1.
1. V1=A, V2=0, V3=A, V4=B, V5=A, V6=1, V7=0, Mass flow regulator (A5): 20 ml/min, Pump (A9): 20 mbar, The system was flushed with the carrier gas.
2. V2=1, The reduction reactor (A1) was flushed for 5 min with H₂ 20 ml/min.
3. V1=B, V2=0, The [¹¹C]carbon dioxide was transferred from the cyclotron to the reduction reactor (A1).
4. V1=A, V2=1, The reactor (A1) was flushed for 2 min with H₂ 20 ml/min
5. V2=0, V3=B, The reactor (A1) was closed
6. The reactor (A1) was heated to convert [¹¹C]carbon dioxide to [¹¹C]methane
7. CH₃I-trap (A8), (teflon tubing id 1.58 mm, od 3.18 mm) was cooled with liquid nitrogen.
8. V4=A, V5=B, The plasma reactor (A7) was turned on. The iodine feed (A6) containing 50 mg anhydrous 10 mesh iodine beads 99.999% (Aldrich) was connected.
9. V3=A, The heating of the reduction reactor (A1) was turned off when it reached 360° C. The reactor was opened and [¹¹C]methane was transferred through the plasma reactor (A7)
10. Paused until [¹¹C]methyl iodide was collected at the CH₃I-trap (A8).
11. V4=B, V5=A, V6=0, The plasma reactor (A7) was turned off and the pump (A9) was disconnected
12. Pause until the pressure prior and after the mass flow controller (A5) was equalized.
13. V7=1, The CH₃I-trap (A8) was heated with warm water and the [¹¹C]methyl iodide was transferred to the vial (A10).

The following equipment was used in the experiments: Valves: V1, V3, V4, V5 EHMA (Vici Valco, US), V2, V6, V7 5300 (Rheodyne, US), Mass flow controller MC-100SCCM (Alicat Scientific, US), Plasma power supply 400V, 31 kHz: DC-AC Inverter 24V S24556 (Miyata Elevam, Japan), Quarts plasma reactor: OZS-0613 Type B (Miyata Elevam, Japan), Vacuum pump N85.3KTDC, (KNF Neuberger, Germany) and PC 2001 Vario (Vacuubrand, Germany). Columns (Onmifit, id 10 mm, length 50 mm), Analytical HPLC was performed on a Beckman system, equipped with a Beckman 126 pump, a Beckman 166 UV detector in series with a Bioscan β⁺-flow count detector and a Waters Spherisorb 5 μm ODS1 column (250×4.6 mm). A Gilson 231 was used as auto injector. Purification with preparative HPLC was performed on a similar Beckman system equipped with a Beckman Ultrasphere ODS dp 5μ column (250×10 mm).

### Characterization of [¹¹C]Methyl Iodide and Determination of Specific Radioactivity

[¹¹C]Methyl iodide was transferred in a stream of helium (20 ml/min) from the CH₃I-trap to a solution of acetonitrile (400 μl, −20° C.). The radioactivity in the vial and the volume of the acetonitrile solution was measured. The concentration of [¹¹C]methyl iodide was determined with analytical HPLC using a standard curve. A) water, 0.1% formic acid; B) acetonitrile (50:50). Flow 1.0 ml/min. [¹¹C]Methyl iodide R.t. 6.0 min. [¹¹C]Methylene iodide R.t. 8.8 min.

### Synthesis of [¹¹C]Flumazenil

N-desmethyl flumazenil (1.0 mg, 3.5 μmol) was added to a solution of dimethylformamide (250 μl) and dimethyl sulfoxide (60 μl) in a 0.8 ml glass vial equipped with a rubber septum. Potassium hydroxide (0.8 μl, 5 M) was added and the resulting solution was vortexed. The [¹¹C]methyl iodide was transferred to the solution in a stream of helium gas (20 ml/min). The vial was heated for 3 min at 75° C. Preparative HPLC: A) 0.05 M ammonium formate pH 3.5; B) acetonitrile/water 50/7; (64:36). Flow 4 ml/min. R.t. 7.4 min. Analytical HPLC was used to assess the identity and radiochemical purity. A) 0.05 M ammonium formate pH 3.5; B) acetonitrile/water 50/7; (60:40). Flow 1.0 ml/min. R.t. 8.6 min.

General Description of Obtaining [¹¹C]Methyl Iodide

A system was developed based on the chemistry route wherein [¹¹C]Methyl iodide was obtained in 13±3% decay corrected radiochemical yield in less than 6 min from [¹¹C]carbon dioxide. Hydrogen and nickel were used in the reduction of [¹¹C]carbon dioxide at 360° C. The formed [¹¹C]methane was converted to [¹¹C]methyl iodide in the system by a single pass through a non-thermal plasma reactor. The plasma was created by applying high voltage to electrodes in a gas stream with regulated pressure and mass flow. The plasma reactor is turned on and off in a similar way as a fluorescent lamp and generates no heat.

### Example 1

### Experimental Studies

[¹¹C]Methyl iodide was prepared from cyclotron-produced [¹¹C]carbon dioxide via nickel catalyzed reduction and iodination initiated by electron impact. Non-thermal plasma has non-equilibrium properties. While the gas temperature in the plasma may be close to room temperature, the free electrons can reach energies up to 10 eV. The electrons are accelerated in an electric field and collisions with molecules initiate chemical reactions. The non-equilibrium property of non-thermal plasma explains why these reactions can occur at low temperature. To reach the same electron energy with plasma in equilibrium (thermal plasma), the temperature of the gas volume needs to be about 360° C. In thermal plasma the energy is divided equally between electrons, ions and neutral particles which may lead to breakdown of thermally unstable reactants or product. Although the gas retains low temperature in non-thermal plasma, a mixture of radicals, excited species and ions are formed, often resulting in low product selectivity. This problem may be overcome by selecting proper reagents and reaction conditions as was nicely demonstrated by Okumoto et al.

In this experiment glow discharge plasma was utilized. This type of non-thermal plasma is generated by applying a high voltage to electrodes in a gas with low pressure. The plasma was sustained with 400 V AC and the power consumption was lower than 6 W. The reduced pressure enabled homogenous excitation of the gas volume in the reactor which then emitted light. The reactor generated small amounts of heat and the plasma could instantly be turned on or off.

Drawings of the current invention are presented in FIGS. 1, 2, and 3.

It could be shown that when the pump is positioned on-line as depicted in FIG. 1, that the total production time would be quicker by another two to three minutes since the on-line pump eliminates the need of an CH₃I-trap. The [¹¹C]methyl iodide would then be transferred through the pump and directly to a vial.

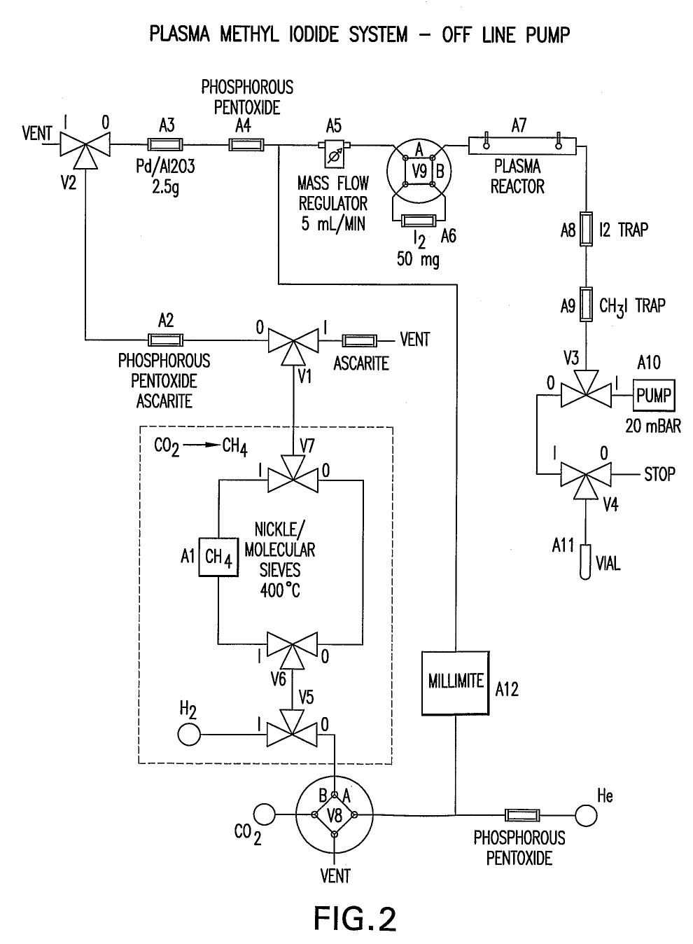

FIGS. 1 and 2. The Experimental Set-Up Used in the Experiments

A mass flow controller (A5) was positioned at the start of the low pressure part which contained the iodine feed (A6), the plasma reactor (A7) and the CH₃I-trap (A8). A diaphragm vacuum pump (A9) was used to reduce the pressure in the plasma reactor.

[¹¹C]Methane was transferred to the plasma reactor by passing through the iodine feed, thus mixing with the iodine before entering the plasma reactor. The entrance of iodine in the plasma field was clearly visible as a change from the inherent colour of the carrier gas to a thick white glow. The high energy electrons in the plasma initiated the reaction which converted [¹¹C]methane to [¹¹C]methyl iodide. The mechanism of the reaction has not yet been studied. However, we assumed that free radicals of iodine were formed in the plasma reacting with [¹¹C]methane. It was not clear if the electron energy was high enough to directly ionize methane.

The formed [¹¹C]methyl iodide was trapped on Teflon-tubing immersed in liquid nitrogen. The vacuum pump was disconnected when the entire batch of radioactivity had passed through the plasma reactor and the system was equilibrated to atmospheric pressure by use of the mass flow controller. The Teflon tubing used as the trap was warmed to room temperature to release the [¹¹C]methyl iodide which then was transferred by the carrier gas to a vial with dimethylformamide (300 μl) where it was analyzed or used in methylation reactions.

Furthermore, both neon and helium were tested as carrier gases. Best results were obtained using helium with the vacuum pump operated at 20 mbar and the mass flow through the plasma reactor regulated to 20 ml/min. When using these conditions [¹¹C]methyl iodide was obtained with a decay corrected radiochemical yield of 13±3% (n=12) based on the amount of [¹¹C]carbon dioxide at start of synthesis. The radiochemical purity was 64±7% (n=5). By inserting a column with phosphorous pentoxide between the CH₃I-trap (A8) and the vial (A10), the radiochemical purity was increased to 88±7% (n=6). The procedure to convert [¹¹C]carbon dioxide and transfer the formed [¹¹C]methyl iodide to the vial required less than 6 min. The conversion yield of [¹¹C]carbon dioxide to [¹¹C]methane was in the order of 90%.

Radionuclide productions using 12 μAh were made when the specific radioactivity of the [¹¹C]methyl iodide was determined. 24 GBq of [¹¹C]carbon dioxide was transferred to the reduction reactor. In the end of the reaction sequence 2.0±0.1 GBq and 4.9±0.6 nmol of [¹¹C]methyl iodide was trapped in acetonitrile at −20° C. The specific radioactivity was 412±32 GBq/μmol (n=2).

Additionally, [¹¹C]Flumazenil was synthesized via methylation of N-desmethyl flumazenil using the produced [¹¹C]methyl iodide. After preparative HPLC-purification, [¹¹C]flumazenil was obtained in 12+t 3% decay-corrected radiochemical yield based on [¹¹C]carbon dioxide (n=3). The [¹¹C]methyl iodide was used as received from the CH₃I-trap. The alkylation reaction proceeded without further purification of the [¹¹C]methyl iodide hence neither a phosphorous pentoxide column nor an iodine trap was used.

The intensity of the plasma was identified as an important factor when the reaction conditions were optimized. As the pressure was decreased in the reactor, the plasma glow became brighter. However, an increase in the intensity of the plasma clearly favoured the formation of ¹¹C-labelled polar by-products, observed in the front of the analytical HPLC chromatogram, and to some extent [¹¹C]methylene iodide. The polar products may be derived from reactions with oxygen which efficiently oxidise methane in the plasma environment. The oxygen may have originated from the target gas or from air leakage in the low pressure part of the system.

The formation of [¹¹C]methylene iodide was also dependent on the amount of iodine entering the plasma reactor, which in turn was affected by the flow rate of the carrier gas, the pump pressure, the amount of iodine in the iodine feed and the inner diameter and length of the tubing (peek id 0.5 mm, length 135 mm) connecting the iodine feed with the plasma reactor. Too high iodine concentration resulted in the formation of [¹¹C]methylene iodide.

Other parameters which affected the efficiency of the plasma reaction were the type of carrier gas and the mass flow rate of the carrier gas through the plasma reactor. A low mass flow resulted in deposition of radioactive material in the reactor. A high mass flow lowered the radiochemical yield due to the weakening of the plasma induced by a pressure increase in the reactor. When the carrier gas was changed from helium to neon the radiochemical purity of [¹¹C]methyl iodide was decreased to approximately 50% and several radiochemical by-products were detected although not yet identified. This could be explained by an increase in the electron density of the plasma due to the lower ionization energy of neon.

The flow of the carrier gas at reduced pressure facilitated the release of a sufficient amount of iodine vapours from the source of 50 mg of iodine. The iodine feed was connected to a switch valve. It enabled the disconnection of the iodine from the gas stream and thus reducing the iodine consumption as well as minimizing problems with contamination in the plasma reactor. Iodine deposited on the walls of the reactor during the reaction was removed by running the plasma without the iodine feed in between the experiments. It was possible to run over 30 experiments on a single load of 50 mg of iodine.

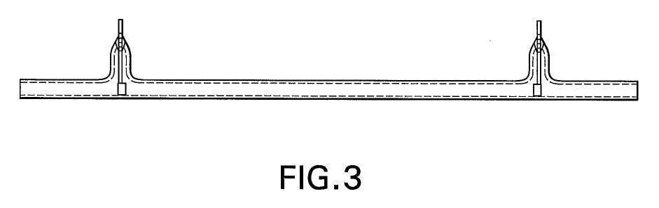

FIG. 3. Drawing of the Plasma Reactor (mm).

The plasma was created by applying 400 V/31 kHz to the electrodes positioned in the gas stream inside the plasma reactor, shown in FIG. 4. The electrodes were made of tungsten and the reactor body was made from quarts glass.

Palladium on solid support is known to efficiently absorb hydrogen gas. The initial experiments were carried out without a hydrogen trap which led to a visible quenching of the plasma glow when the [¹¹C]methane was passing through the plasma reactor. The glow plasma was sustained to a high degree when a column with palladium on aluminium oxide was inserted upstream from the plasma reactor. Water which was formed in the reaction between oxygen and the palladium-hydride complex was removed on a column containing phosphorous pentoxide inserted after the hydrogen trap.

Glow discharge plasma was selected for this study due to the uncomplicated and inexpensive reactor and electrical components needed to generate the plasma. The low pressure was favourable for the release of iodine to the reactor. There are several other types of techniques to create non-thermal plasma which is interesting in the context of ¹¹C-labeling chemistry. Dielectric barrier discharge plasma can be sustained at atmospheric pressure. The plasma is generated by applying high voltage pulses to an electrode which is shielded from the grounded electrode by a dielectric barrier, e.g. quartz. Micro-discharges occur in the gas volume in between the electrode and the dielectric barrier. The high voltage pulse can be designed with respect to the application by changing the voltage, frequency and pulse rise time etc. Two other ways of creating non-thermal plasma is to use microwave discharges and radio-frequency discharges which have the advantage that the reagents entering the plasma are not in contact with the electrodes.

We are now exploring alternative techniques for efficient production of [¹¹C]methyl iodide using non-thermal plasma in single-pass and recirculating systems. Especially dielectric discharge plasma is of interest since it allows the ionizing strength of the plasma to be adjusted and to sustain the plasma at atmospheric pressure. The problem associated with leaks and air entering the reactor may be eliminated when using systems operated at atmospheric pressure.

II. Plasma Methyl Iodide System—Off Line Pump

The parameters for the experimental set-up outlined for FIG. 2 are as follows:

A drawing of the plasma reactor used is presented in FIG. 3.
Carrier gas: Helium
Pump pressure: 5 mbar
Mass flow regulator set to 20 ml/min
400V, 31 kHz plasma power supply
Iodine load: 50 mg I₂,
Hydrogen trap: 2.5 g Pd/Al₂O₃
(Flush the System with Helium)
14. V8=A, V9=A
15. V1=0, V2=0, V5=0, V6=0, V7=0
16. V4=0, V3=1
17. Pump on (A10)
18. Open millimite valve (A12) (Fine control valve with micrometer hand wheel)
(Flush the methane reactor (A1) with hydrogen gas)
19. V1=1, V6=1, V7=1
20. V5=1
21. H₂ 20 ml/min approximately 5 min.
(Convert [¹¹C]carbon dioxide to [¹¹C]methane)
22. V8=B
23. V5=0
24. Release activity from cyclotrone
25. [¹¹C]carbon dioxide is trapped in methane reactor (A1)
26. V8=A
27. V5=1
28. Wait 2 minutes while the methane reactor (A1) is flushed with H₂
29. V6=0, V7=0, V5=0
30. Cool the CH₃I-trap (A9) with liquid nitrogen
31. Heat methane reactor (A1) to convert [¹¹C]carbon dioxide to [¹¹C]methane
32. When the reactor reaches 400° C., turn the plasma on (A7)
33. V9=B Turns the iodine feed (A6) on
34. Turn the millimite off (A12)
35. V1=0,
36. V6=1, V7=1 [¹¹C]methane is released from the methane reactor (A1).
([¹¹C]methane is released and passed trough the plasma reactor (A7) where it is converted to [¹¹C]methyl iodide.)
37. Trapping finished of [¹¹C]methyl iodide at the CH₃I-trap (A9).
38. V9=A, Iodine feed off. (A6)
39. Turn off plasma (A7)
40. V6=0, V7=0, V3=0
41. When pressure difference prior and after the flow regulator (A5) is equalized. V4=1
42. Heat MeI-trap (A9) to release the trapped [¹¹C]methyl iodide
43. [¹¹C]Methyl iodide is trapped in vial (A1)

### Proof of Principles:

By using the above method described, the following result was achieved:

[¹¹C]methyl iodide was produced with a decay corrected radiochemical yield of 12% based on the amount of [¹¹C]carbon dioxide used in the production of [¹¹C]methane. The radiochemical purity was 99%. The procedure took 6.5 min.

The yield obtained by the current invention is 3-4 times higher compared with a single pass “gas phase methyl iodide” system. Therefore there is great potential for the plasma system to supersede the “gas phase” by using a CH₃I trap to take out [¹¹C]methyl iodide and recirculate the [¹¹C]methane back into the plasma reactor. The higher yield per cycle would suggest that a shorter cycle time compared with the “gas phase” method is needed.

### Specific Embodiments

### CITATION OF REFERENCES

The present invention is not to be limited in scope by specific embodiments described herein. Indeed, various modifications of the inventions in addition to those described herein will become apparent to those skilled in the art from the foregoing description and accompanying figures. Such modifications are intended to fall within the scope of the appended claims.

Various publications and patent applications are cited herein, the disclosures of which are incorporated by reference in their entireties.

What is claimed is:

1. A method for preparing [¹¹C]methyl iodide in a plasma reactor system, comprising the steps of:
obtaining methane (A1) from the reduction of carbon dioxide with nickel
and then adding a cyclotron to produce [¹¹C] onto the methane target whereby [¹¹C]methane is carried into said system in a stream of a carrier gas whereby a carbon dioxide trap (A2), a drying tower (A2 and A4), and a hydrogen trap (A3) are used to remove hydrogen;
introducing a mass flow regulator (A5) to mark the start of the low pressure area of said system whereby the low pressure area contains an iodine source (A6), a plasma reactor (A7), and a trapping chamber (A8) and wherein the mass flow regulator is regulated by a pump (A10);
transferring [¹¹C]methane via said iodine source into said plasma reactor, whereby iodine vapors form and pass through said system simultaneously
with [¹¹C]
methane to further form a gas stream;
applying a voltage to electrodes positioned in said gas stream inside said system, whereby electrons are stripped from the carrier gas and plasma is created thus forming a [¹¹C]methyl iodide plasma, and then
transferring said [¹¹C]methyl iodide plasma via said trapping chamber and said pump
to a reaction vial (A11) containing a suitable solvent or a loop reaction system.

2. The method according to claim 1, wherein the low pressure area in the plasma reactor system is in the range from about 5 to about 250 mbar.

3. The method according to claim 1, wherein the drying tower is phosphorous pentoxide.

4. The method according to claim 1, wherein the hydrogen trap is Pd/Al₂O₃.

5. The method according to claim 1, wherein the carrier gas is helium, argon or neon.

6. The method according to claim 1, wherein the carrier gas residence time in the plasma reactor system is in the range from about 0.5-1.0 second.

7. The method according to claim 1, wherein the plasma reactor generates no heat.

8. The method according to claim 1, wherein the iodine source comprises of about 50 mg of I₂.

9. The method according to claim 1, wherein the trapping chamber is a column containing sodium hydroxide on silica that absorbs iodide but not methyl iodide.

10. The method according to claim 1, wherein the trapping chamber comprises an optional CH₃I-trap.

11. The method according to claim 1, wherein the pump is positioned on-line or off-line.

12. The method according to claim 1, wherein the voltage applied to the electrodes positioned in said gas stream is about 400 Volts with a frequency of about 31 kHz.

13. The method according to claim 1, wherein the mass flow regulator has a mass flow of about 1 ml/min to about 50 ml.min.

14. The method according to claim 1, wherein the suitable solvent is dimethyl sulfoxide, N,N-dimethyl formamide, N-methyl pyrollidone, or similar compounds.

15. The method according to claim 1, wherein the loop reaction system is a method for using small amounts of reaction media to trap the [¹¹C]methyl iodide, whereby the reaction media is coated on the internal surface of a piece of tubing and the [¹¹C]methyl iodide is then directed through the tubing to get trapped in the reaction media.

16. The method according to claim 1, wherein the reaction media is [¹¹C]methane.

17. The method according to claim 1, wherein any unreacted [¹¹C]methane is recirculated back into the plasma reactor and the [¹¹C]methyl iodide would be taken out from the recirculation by the use of a methyl iodide trap.

18-29. (canceled)

30. A kit used for preparing a method for producing [¹¹C]methyl iodide, wherein the method comprises the steps of:
obtaining methane (A1) from the reduction of carbon dioxide with nickel
and then adding a cyclotron to produce [¹¹C] onto the methane target whereby the [¹¹C]methane is carried into said system in a stream of a carrier gas whereby a carbon dioxide trap (A2), a drying tower (A2 and A4), and a hydrogen trap (A3) are used to remove hydrogen;
introducing a mass flow regulator (A5) to mark the start of the low pressure area of said system whereby the low pressure area contains an iodine source (A6), a plasma reactor (A7), and a trapping chamber (A8) and wherein the mass flow regulator is regulated by a pump (A10);
transferring [¹¹C]methane via said iodine source into said plasma reactor, whereby iodine vapors form and pass through said system simulataneously simultaneously with [¹¹C]
methane to further form a gas stream;
applying a voltage to electrodes positioned in said gas stream inside said system, whereby electrons are stripped from the carrier gas and plasma is created thus forming a [¹¹C]methyl iodide plasma, and then
transferring said [¹¹C]methyl iodide plasma via said trapping chamber and said pump
to a reaction vial (A11) containing a suitable solvent or a loop reaction system.

31-38. (canceled)

39. The kit according to claim 30, wherein the trapping chamber comprises an optional CH₃I-trap.

40-44. (canceled)

45. The kit according to claim 30, wherein the reaction media is [¹¹C]methane.

46. The kit according to claim 30, wherein any unreacted [¹¹C]methane is recirculated back into the plasma reactor and the [¹¹C]methyl iodide would be taken out from the recirculation by the use of a methyl iodide trap.

47-65. (canceled)
